(19) United States
(12) Patent Application Publication
(10) Pub. No.: US 2014/0303667 A1
(43) Pub. Date: Oct. 9, 2014

(54) EMBOLIC PROTECTION DEVICE AND METHODS OF USE

(71) Applicant: Inceptus Medical, LLC, Aliso Viejo, CA (US)

(72) Inventors: Brian J. Cox, Laguna Niguel, CA (US); Paul Lubock, Monarch Beach, CA (US); Robert Rosenbluth, Laguna Niguel, CA (US); Richard Quick, Mission Viejo, CA (US)

(73) Assignee: INCEPTUS MEDICAL, LLC, Aliso Viejo, CA (US)

(21) Appl. No.: 14/362,376

(22) PCT Filed: Nov. 30, 2012
(86) PCT No.: PCT/US12/67479

§ 371 Date: Jun. 3, 2014

Related U.S. Application Data
(60) Provisional application No. 61/566,531, filed on Dec. 2, 2011.
(60) Provisional application No. 61/646,833, filed on May 14, 2012.

(51) Int. Cl. A61F 2/01

(52) U.S. Cl. CPC A61F 2/01; A61F 2002/011
USPC 606/200

(57) ABSTRACT

Embolic protection devices for placement in an arterial system of a human and methods of using such devices. In one embodiment, the device includes a filter having a first filter section configured to be deployed along the ascending aorta and aortic arch, a tapered second filter section extending proximally from the first filter section, and a proximal portion extending proximally from the second filter section. The second filter section is configured to be deployed along the descending aorta, and the proximal portion extends to a position below a renal artery. The embolic protection device can be configured to continuously filter the arterial system blood between a distal end of the first filter section to a position within the arterial system downstream of the renal artery.

### CROSS-REFERENCE TO RELATED APPLICATIONS

The present application claims priority to U.S. Provisional Application No. 61/566,531, filed Dec. 2, 2011, entitled “EMBOLIC PROTECTION DEVICES AND METHODS,” and U.S. Provisional Application No. 61/646,833, filed May 14, 2012, entitled “EMBOLIC PROTECTION DEVICES AND METHODS,” the full disclosures of which are incorporated herein by reference.

### TECHNICAL FIELD

The present technology relates generally to devices and methods for providing protection against emboli entering the aortic arch vessels (e.g., the brachiocephalic, left common carotid and left subclavian arteries) as well as the downstream vessels branching from the aorta (e.g., the celiac, mesenteric, renal, gonadal and iliac arteries). Many embodiments of the technology relate to protecting patients from emboli created or dislodged by the passage and deployment of transaortic devices, such as Transcatheter Aortic Valve Replacement (“TAVR”) devices, catheters or cannulae.

### BACKGROUND

Procedures involving the placement and delivery of catheters and devices retrograde through the aorta to the heart have led to a growing concern for the potential of neurological complications, such as stroke, and other organ infarction due to emboli created or released by the deployment and use of transaortic devices, transmitral device, catheters, cannulae or other therapeutic devices. Emboli may include calcified plaques, artheromatous fragments, thrombus, fat globules, air or gas bubbles, clumps of bacteria or other foreign material, tissue remnants or tumor cells. The emboli may travel to downstream vessels and cause infarction (i.e., tissue death caused by a local lack of oxygen due to obstruction of the tissue's blood supply).

Embolic material can be created or released during therapeutic procedures because of mechanical trauma, such as abrasion, bumping, bending, torqueing, dilation or expansion of the often atherosclerotic walls of the aorta, calcified heart valves, or other diseased or thrombus containing structures of the cardiovascular system. Other potential causes of emboli may include the physiological stress induced by the procedure and aggravation of underlying conditions such as valve disease, atrial fibrillation and structural heart defects leading to the incidental release of thrombus. Air emboli or particulates released from the device are also potential sources of emboli.

Devices to protect patients from emboli have been proposed or introduced with limited success. Such existing devices are generally placed percutaneously with a catheter through the radial, brachial or subclavian artery, or through the femoral artery to the aortic arch under fluoroscopic guidance. Many existing devices, however, have drawbacks such as merely deflecting or diverting embolic matter as opposed to effectively capturing and retaining emboli, traumatizing the vessel wall, and several other drawbacks explained in more detail below. Accordingly, there is a need for devices and methods that address one or more of these deficiencies.

### BRIEF DESCRIPTION OF THE DRAWINGS

The accompanying drawings, which are incorporated herein and constitute part of this specification, illustrate embodiments of the present technology, and, together with the general description given above and the detailed description given below, serve to explain the features of the present technology.

FIG. 1 is a side perspective view of an expandable embolic protection device in a deployed state (e.g., expanded configuration) for temporary placement in the aorta in accordance with an embodiment of the present technology.

FIG. 1A is an axial cross-sectional view taken from Section A-A of FIG. 1.

FIG. 1B is an axial cross-sectional view taken from Section B-B of FIG. 1.

FIG. 1C is an axial cross-sectional view taken from Section C-C of FIG. 1.

FIG. 2 is a side perspective view of an expandable embolic protection device in a deployed state (e.g., expanded configuration) for temporary placement in the aorta in accordance with an embodiment of the present technology.

FIG. 2A is an enlarged side perspective view of fixation structures at a distal region of an expandable embolic protection device in a deployed state in accordance with an embodiment of the present technology.

FIG. 2B is an enlarged side perspective view of a stent at a distal region of an expandable embolic protection device in a deployed state in accordance with an embodiment of the present technology.

FIG. 3 is a schematic cross-sectional view of one embodiment of a delivery system in accordance with an embodiment of the present technology.

FIG. 4 is a side cross-sectional view of an expandable embolic protection device in a collapsed configuration within the delivery system of FIG. 3 at a target location in the aorta.

FIG. 4A is an enlarged cross-sectional side view of select components at a proximal region of an embolic protection device delivery system in accordance with an embodiment of the present technology.

FIG. 4B is an enlarged cross-sectional side view of select components at a distal region of an embolic protection device delivery system in accordance with an embodiment of the present technology.

FIG. 5 is a perspective side view of a therapeutic device delivered to a target arterial location through a deployed embolic protection device in accordance with an embodiment of the present technology.

FIG. 6 is a retrieval sheath for use with the embolic protection device in accordance with an embodiment of the present technology.

FIG. 7 is an enlarged view of a self-expanding braid with interwoven large and small strands configured in accordance with an embodiment of the present technology.

FIG. 8 is a side view of a mandrel and a braided mesh formed over the mandrel configured in accordance with an embodiment of the present technology.

FIG. 9 is a schematic cross-sectional side view of a mesh comprising two braided layers configured in accordance with an embodiment of the present technology.

FIG. 10 is a schematic cross-sectional side view of a mesh comprising an inner braided layer sandwiched between an everted outer braided layer configured in accordance with an embodiment of the present technology.

### DETAILED DESCRIPTION

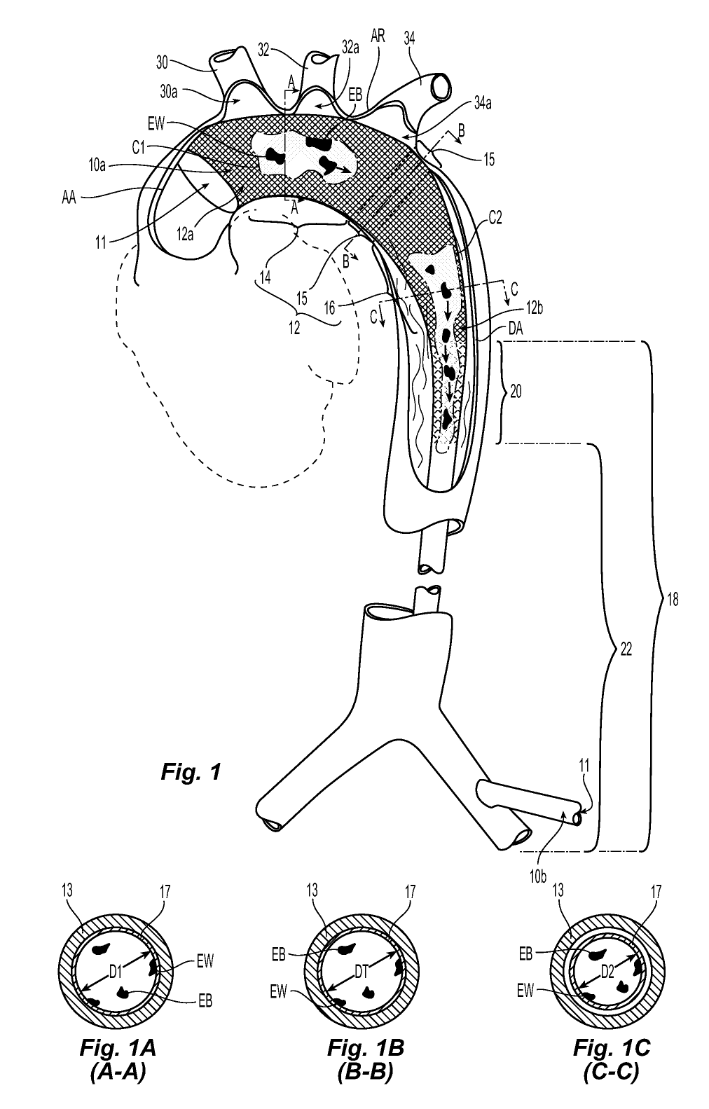

Specific details of several embodiments of the technology are described below with reference to FIGS. 1-10. Although many of the embodiments are described below with respect to devices, systems, and methods for embolic protection, especially during interventional cardiovascular procedures, other applications and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-10.

With regard to the terms “distal” and “proximal” within this description, unless otherwise specified, the terms can reference a relative position of the portions of an embolic protection device and/or an associated delivery device with reference to an operator and/or a location in the vasculature. For example, proximal can refer to a position closer to the operator of the device or an incision into the vasculature, and distal can refer to a position that is more distant from the operator of the device or further from the incision along the vasculature. For ease of reference, throughout this disclosure identical reference numbers are used to identify similar or analogous components or features, but the use of the same reference number does not imply that the parts should be construed to be identical. Indeed, in many examples described herein, identically numbered parts of individual embodiments are distinct in structure and/or function. The headings provided herein are for convenience only.

With regard to anatomical terminology, most patients have three arteries branching off the aortic arch: the brachiocephalic artery ostium, left common carotid artery ostium, and left subclavian artery ostium. However, in a minority of patients, the left common carotid artery ostium and the left subclavian artery ostium may be merged such that the patient effectively only has two arteries branching from the aortic arch.

Systems, devices and methods provided herein for protecting against emboli entering the aortic arch vessels and/or other downstream vessels branching from the aorta in accordance with several embodiments of the technology are generally used during minimally invasive cardiac procedures. In one embodiment, the embolic protection device has a low-profile configuration (i.e., undeployed) for delivery through the vasculature and an expanded configuration (i.e., deployed) for temporary placement within a patient's arterial system. The embolic protection device may include a filter portion comprising a mesh configured to allow sufficient blood flow through the vasculature while retaining emboli for eventual removal from the patient. In some embodiments, the embolic protection device may further include a proximal portion attached to or integrated with the filter portion, and the proximal portion extends proximally (e.g., downstream) from the filter portion. In the expanded configuration, the filter portion can have a first filter section with a first cross-sectional dimension configured to anchor the embolic protection device at least within the ascending aorta at a location distal to the brachiocephalic artery ostium. The first filter section can have a length and cross-sectional dimension configured to cover the ostia of the aortic arch vessels in a manner that prevents emboli from entering the aortic arch vessels while allowing sufficient blood flow through the ostia. In selected embodiments, the filter may also include a tapered second filter section extending proximally from the first filter section. The tapered second filter section, for example, may start tapering from the first filter section at a point along the length of the device near or downstream of the left subclavian artery ostium. The filter may include one or more layers of self-expanding meshes (e.g., braided material), and the proximal portion may extend proximally (e.g., downstream) from the second filter section of the filter to an extracorporeal location.

Several embodiments of the technology include devices and methods wherein at least a portion of emboli in the bloodstream are deflected from entering a branch vessel of the aorta, and then captured within a space between the embolic protection device and the therapeutic device downstream of the aortic arch. Several embodiments, for example, continue to capture and deflect emboli in the blood stream through the descending aorta.

### 1. Selected Embodiments of Embolic Protection Devices

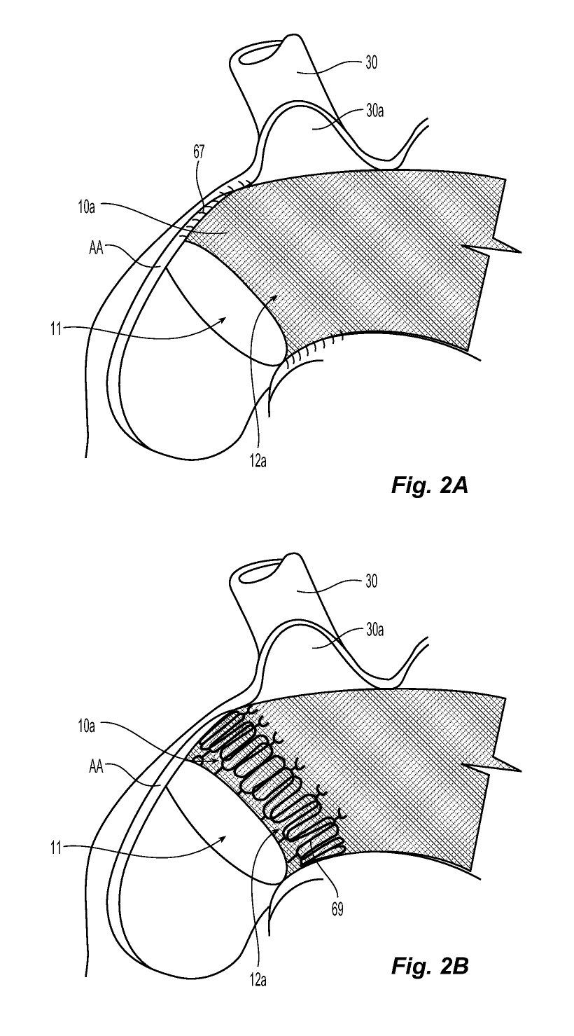

FIG. 1 is a side perspective view of one embodiment of an embolic protection device (EPD) 10 in an expanded configuration within the aorta. The EPD 10 can have a distal region 10a temporarily anchored within the ascending aorta AA (distal the brachiocephalic artery ostium 30a), a proximal region 10b located at an extracorporeal location 24, and a longitudinal dimension between the distal region 10a and the proximal region 10b. The EPD 10 can have a lumen 11 extending from the distal region 10a to the proximal region 10b. In the embodiment shown in FIG. 1, the EPD 10 further includes a filter portion 12 and a proximal portion 18 extending proximally the filter portion 12 to the proximal region 10b of the device. Therefore, emboli entering the distal region 10a of the EPD 10 are permanently removed from the arterial system rather than merely being deflected downstream of the aortic arch, as is the case in many existing devices. For example, the emboli EW may be filtered/trapped/retained anywhere along a wall 17 of the filter portion 12 (see FIGS. 1A-1C) and subsequently removed with the EPD 10 at the end of the procedure, and/or free floating emboli EB may be funneled through the lumen 11 of the EPD 10 to a proximal hub 72 of the delivery catheter (see FIG. 3) and/or outside of the patient. The arterial span of the EPD 10 not only prevents the emboli from entering the aortic arch vessels (brachiocephalic artery, left common carotid artery, and the left subclavian artery), but also from entering any arterial vessel downstream of the ascending aorta 36 such as the renal, celiac, and mesenteric arteries.

As shown in FIG. 1, the filter portion 12 has a distal zone 12a at an upstream area of the vessel and a proximal zone 12b at a downstream area that define a filter length along a longitudinal dimension. The filter length, for example, can be at least 4 inches in a deployed state. The filter portion 12 can include at least one mesh layer made from a flexible, self-expanding material that can adjust and conform to a dynamic inner lumen of the aorta at a target location to temporarily secure the EPD 10 to the aortic lumen. The distal zone 12a can also be configured to seal the filter portion 12 to the arterial wall 13 at least at an area of the vessel distal to the brachiocephalic artery ostium 30a. Many existing devices are unable to conform to the inner anatomy of the aorta and may be dislodged because of the dynamic fluid conditions of the cardiovascular system, anatomical variations of the aortic arch, presence of atherosclerotic plaque and other reasons. Consequently, many existing devices may not provide adequate sealing against the aorta allowing emboli leakage.

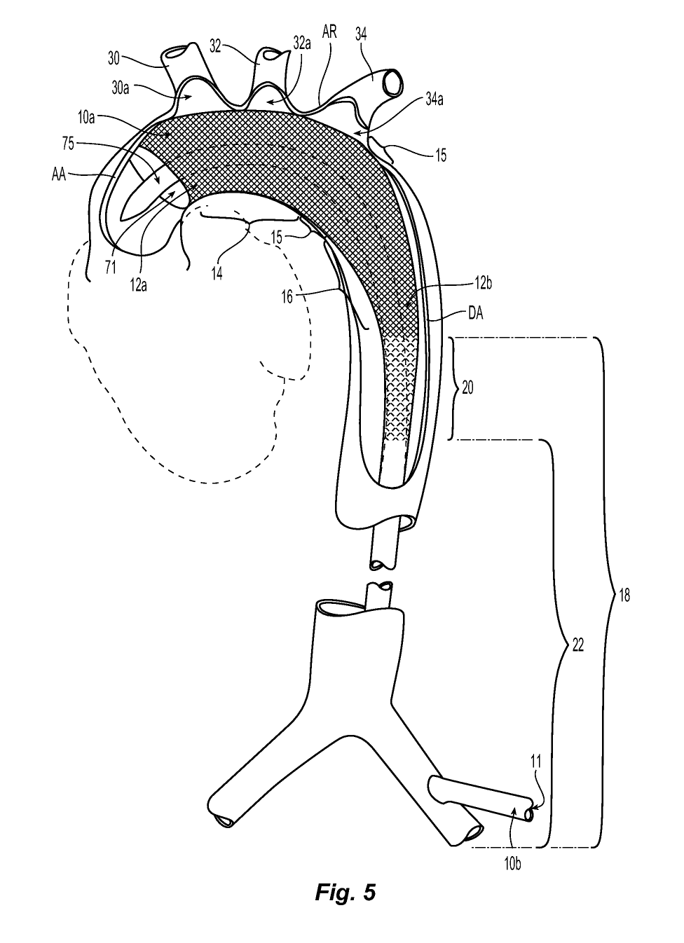

The mesh layer of the filter portion 12 may comprise a braided mesh of filaments (e.g., wires, threads, sutures, fibers, etc.) that have been configured to form a porous fabric or structure for collecting and retaining emboli while simultaneously allowing passage of filtered blood through the vasculature, as shown in cut-away areas C1 and C2 of FIG. 1. The mesh layer, for example, can be fabricated to have a specific shape, porosity, weave density and/or braid angle configured to mitigate disruption to the arterial flow while capturing and removing a large spectrum of emboli. The present technology, for example, can use braids with higher filament counts and smaller pore sizes than current embolic protection devices such that the EPD 10 captures and retains emboli along its length instead of merely diverting emboli away from the vessels branching from the aortic arch. As described below with reference to FIG. 7, “pore size” refers to the diameter of the largest circle 94 that fits within an individual cell of a braid. For example, the at least one mesh layer may have a pore size from about 0.03 mm to about 0.50 mm in selected regions or along its entire length. In selected embodiments, the pore size may be from about 0.060 mm to about 0.25 mm, or from about 0.10 mm to about 0.20 mm. In some embodiments, regions of the filters can have braided filaments having weave densities ranging between 25-75% (discussed below). Such small pore size and high weave count may obviate the need for polymer fabric components that can increase thromboembolic risk due to clot formation.

Referring to FIG. 1, the filter portion 12 may further include a first filter section 14, a transition region 15, and a second filter section 16. The first filter section 14, transition region 15, and the second filter section 16 can be regions of the same mesh material (e.g., a single integral braid with a constant or varying pore size), or they can be separate mesh components that are fused or otherwise connected together (e.g., two or more different braids). A distal terminus of the first section 14 can coincide with or is at least proximate the distal end of the distal region 10a of the EPD 10, and the first filter section 14 of the filter portion 12 extends proximally from the distal region 10a to the transition region 15. The length of the first filter section 14 can be selected such that the transition region 15 is located near the left subclavian ostium 34a when deployed, and in many embodiments the length of the first filter section 14 is selected such that the transition region 15 is downstream of the left subclavian ostium 34a. The distal region 10a of the EPD 10 at the distal terminus of the first filter section 14 is configured to expand to have a first cross-sectional dimension D1 (FIG. 1A) such that the first filter section 14 contacts at least a portion of the ascending aorta AA distal the brachiocephalic ostium 30a and allows for ingress/egress of a therapeutic device through the distal region 10a of the EDP 10. As such, emboli originating upstream of the distal region 10a of the EPD 10 are contained within the filter portion 12 to protect the aortic arch vessels (and depending organs) from emboli in the bloodstream.

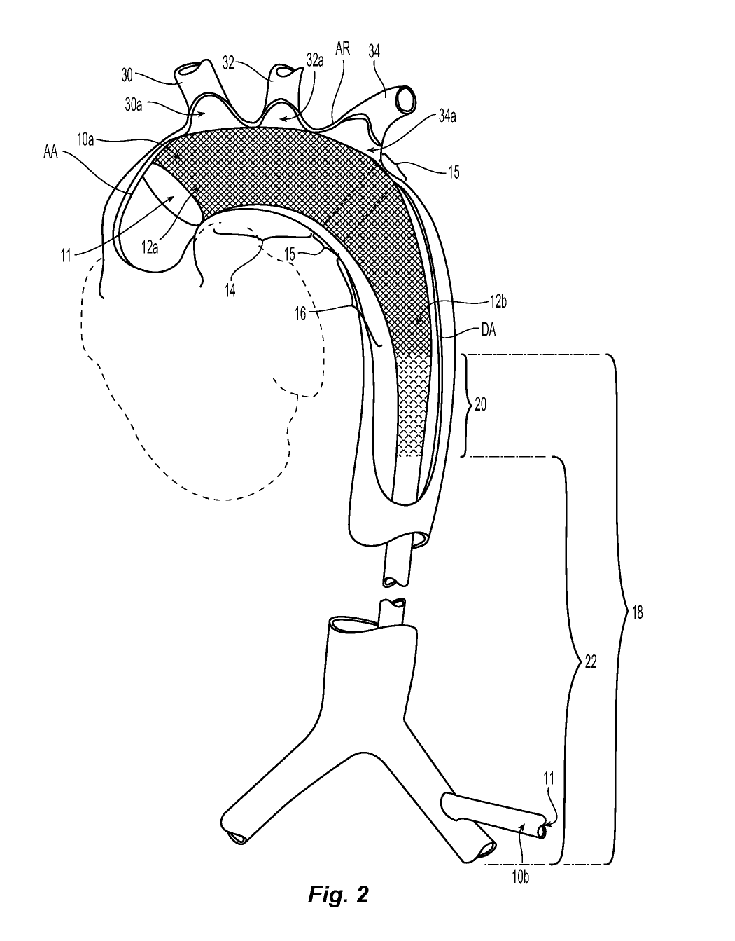

As illustrated in FIG. 1, the first filter section 14 can have a generally cylindrical shape that exerts a contact force (i.e., radial and/or frictional) against the entire circumference of the arterial wall 13 for a length that provides additional anchoring to secure the EPD 10 at a target location. Several existing devices contact the full circumference of the aorta for only on a relatively short distance distal to the brachiocephalic ostium. This approach may be problematic, however, as a shorter anchoring area is less stable and is more likely to traumatize the arterial wall as the forces exerted by the EPD 10 are concentrated at a single, small area within the aorta. In the embodiment of the present technology shown in FIG. 1, the contact force(s) exerted by the first filter section 14 against the arterial wall are distributed along the length of first filter section 14 from the ascending aorta AA to an area downstream of the left subclavian artery ostium 34a, rather than only at a relatively short section of the aorta. This mitigates trauma to the arterial walls. In other embodiments, the first filter section 14 may not contact the full inner circumference of the aorta distal of the left subclavian artery ostium 34a, but rather the first filter section 14 may be spaced apart from the lower portion of the vessel wall through the aortic arch, as shown in FIG. 2.

The second filter section 16 extends proximally from the transition region 15 to the proximal end 12b of the filter portion 12. When deployed, a second filter section 16 may have a second cross-sectional dimension D2 (FIG. 1C) that decreases in a proximal (e.g., downstream) direction such that the second filter section 16 is tapered. For example, a cross-sectional dimension DT of the transition region 15 can be greater than the second cross-sectional dimension D2 at the proximal zone 12b of the filter portion 12. Such a tapered shape facilitates retrieval of the EPD 10. In other embodiments, the second filter section 16 can be generally cylindrical and expand to a cross-sectional dimension D2 such that the second filter section 16 contacts the arterial wall along its length.

The length of the second section 16 can be selected such that, when deployed, the proximal zone 12b of the filter portion 12 may be located within the descending aorta DA or at any point along the aorta downstream of the left subclavian ostium 34a (e.g., the thoracic aorta, the abdominal aorta, the iliac branch, or the femoral artery). In some embodiments, the second section 16 of the filter portion 12 may have a length that extends from the transition region 15 to an inner diameter of an access site introducer sheath and/or an extracorporeal location 24.

Referring to FIG. 1, the proximal portion 18 of the EPD 10 can be attached to or integrated with the filter portion 12. The proximal portion 18 extends proximally from the filter portion 12 to the proximal region of the EPD 10b. The proximal portion 18 may comprise at least one of a coated mesh section 20 and/or an expandable tube section 22. The coated mesh section 20, for example, may be a self-expanding braid and comprise at least one braid layer embedded or covered with a polymeric material by molding, insert molding, dipping, spraying, bonding or other fabrication methods known in the art. The expandable tube section 22 can be a continuous solid wall material that expands when deployed and may be made of polymers or metals including, e.g., Dacron®, polyester, polypropylene, nylon, Teflon, PTFE, ePTFE, TFE, PET, TPE, silicone, polyurethane, polyethylene, ABS, polycarbonate, styrene, polyimide, PEBAX, Hytrel, poly vinyl chloride, HDPE, LDPE, PEEK, rubber, latex as well as Nitinol, platinum, cobalt-chrome alloys, 35N LT, Elgiloy, stainless steel, tungsten, titanium and others.

As shown in FIG. 2A, in some embodiments of the device, the distal region 10a of the EPD 10 may incorporate fixation structures 67 to further secure the EPD 10 to the inner wall of the vessel. The fixation structures 67 may include, e.g., at least one tine, barb, hook, pin or anchor. The fixation structures may have a length of about 0.5 to 3 mm and span about 1 to 2 cm along a length of the distal region 10a. In another embodiment, the length of the fixation structure(s) may be 2-5 mm. Referring to FIG. 2B, the EPD 10 may also include other structures for support, anchoring and/or expanding the distal region 10a, such as additional expandable wires, struts, supports, clips, springs, inflatable balloons, toroidal balloons, glues, adhesives. For example, the EPD 10 can have a stent or scaffold 69 at the distal region 10a. The EPD 10 may further include a vacuum at the distal region 10a. These additional support structures and/or a vacuum may be coupled to the distal region 10a of the EPD using suturing or mechanical coupling techniques known in the art.

Optionally, the embolic protection device may be constructed to elute or deliver of one or more beneficial drug(s) and/or other bioactive substances into the blood or the surrounding tissue. In some embodiments, one or more eluting filament(s) may be interwoven into the mesh to provide for the delivery of drugs, bioactive agents or materials with a mild inflammatory response as disclosed herein. The interwoven filaments may be woven into the mesh structure after heat treating (as discussed below) to avoid damage to the interwoven filaments by the heat treatment process.

Optionally, the device may be coated with various polymers to enhance its performance, fixation and/or biocompatibility. Optionally, the device may incorporate cells and/or other biologic material to promote sealing, reduction of leak or healing.

In any of the above embodiments, the device may include a drug or bioactive agent to enhance the performance and/or healing of the device, including: an antiplatelet agent, including but not limited to aspirin, glycoprotein IIb/IIIa receptor inhibitors (including, abciximab, eptifibatide, tirofiban, lamifiban, fradafiban, cromafiban, toxifiban, XV454, lefradafiban, klerval, lotrafiban, orbofiban, and xemilofiban), dipyridamole, apo-dipyridamole, persantine, prostacyclin, ticlopidine, clopidogrel, cromafiban, cilostazol, and nitric oxide.

In any of the above embodiments, the device may include an anticoagulant such as heparin, low molecular weight heparin, hirudin, warfarin, bivalirudin, hirudin, argatroban, forskolin, ximelagatran, vapiprost, prostacyclin and prostacyclin analogues, dextran, synthetic antithrombin, Vasoflux, argatroban, efegatran, tick anticoagulant peptide, Ppack, HMG-CoA reductase inhibitors, and thromboxane A2 receptor inhibitors.

### 2. Delivery Systems and Methods

FIGS. 3-5 illustrate embodiments of a delivery system 60 and methods for deploying the EPD 10. FIG. 3 is a cross-sectional side view of one embodiment of the delivery system 60 showing the EPD 10 in a collapsed, low-profile configuration for percutaneous delivery. The delivery system 60 may include a guidewire 68 and a single or multi-lumen delivery catheter 70 having a proximal hub 72 and a sheath 73. The sheath 73 has a distal zone 70a, a proximal zone 70b, and a lumen therethrough. For example, the lumen of the sheath 73 can have a diameter between 6F and 30F. The delivery system 60 can further include an obturator 74 having a tapered distal end portion 74a (optional) received within the lumen of the sheath 73 to protect and secure the EPD 10 in its collapsed configuration during delivery.

Access to the ascending aorta or other vessels of the heart can be accomplished through the patient's vasculature in a percutaneous manner. By percutaneous it is meant that a location of the vasculature remote from the heart is accessed through the skin, typically using a surgical cut down procedure or a minimally invasive procedure, such as using needle access through, for example, the Seldinger technique. The ability to percutaneously access the remote vasculature is well-known and described in the patent and medical literature. Once percutaneous access is achieved (for example, through the femoral or iliac arteries), the interventional tools and supporting catheter(s) may be advanced to the heart intravascularly and positioned within the aorta in a variety of manners, as described herein.

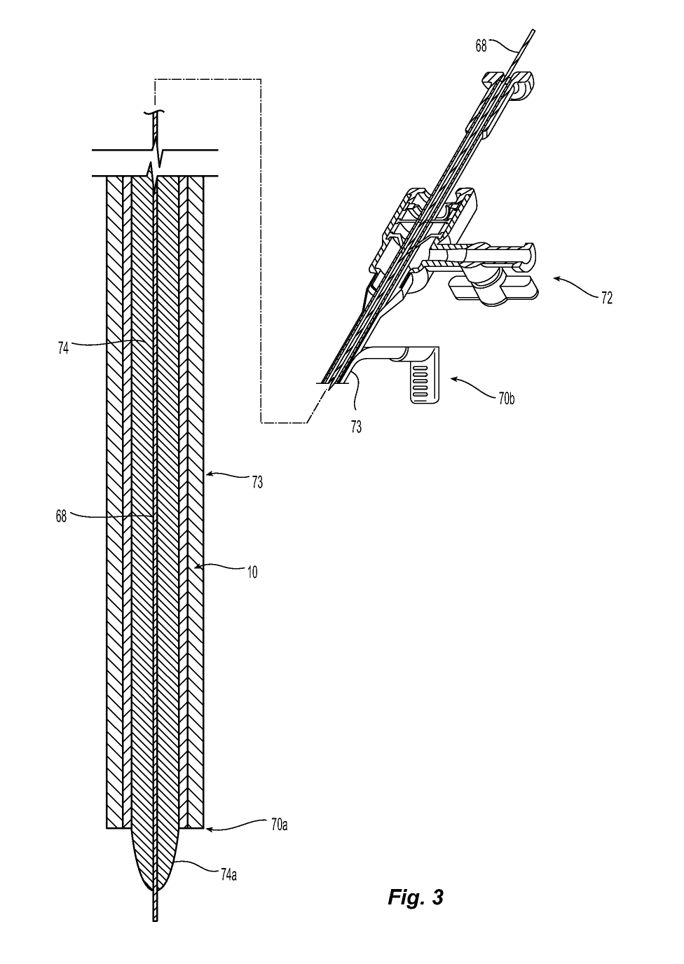

FIGS. 4-4B illustrate one example for delivering and deploying an EPD 10 and/or one or more interventional devices using a retrograde approach. As shown in FIG. 4, a guidewire may be advanced intravascularly within the ascending aorta AA to an area distal to the brachiocephalic artery ostium 30a. The delivery sheath 73, collapsed EPD 10 and obturator 74 can be advanced together over the guidewire 68 until the distal zone 70a is positioned at a target location. The guidewire 68 and catheter 70 can be advanced through the vasculature using known imaging systems and techniques such as fluoroscopy, x-ray, MRI or the like. Radiopaque markers (not shown) can be incorporated into the guidewire 68, catheter 70, or the EPD 10 itself to provide additional visibility under imaging guidance. Such marker materials can be made from tungsten, tantalum, platinum, palladium, gold, iridium, or other suitable materials.

After the distal zone 70a of the delivery catheter 70 is at a target location in the ascending aorta at a location distal of the brachiocephalic artery ostium 30a, the guidewire 68 and obturator 74 are removed proximally (e.g., downstream) through the lumen of the delivery catheter 70. Next, the sheath 73 is retracted proximally and an exposed portion of the EPD 10 expands (FIG. 4B) such that a portion of the EPD 10 contacts the arterial wall distal the brachiocephalic artery ostium 30a. In some embodiments, the EPD 10 may be actively expanded using conventional techniques known in the art, such as pull-wires attached to a distal end of the device and/or a balloon assembly. As shown in FIG. 4A, the sheath 73 may be a “split sheath” that separates into two or more parts at the proximal zone 70b as it is moved proximally (FIG. 4A). In other embodiments, the sheath 73 may be retracted using pull-wires attached to the sheath 73.

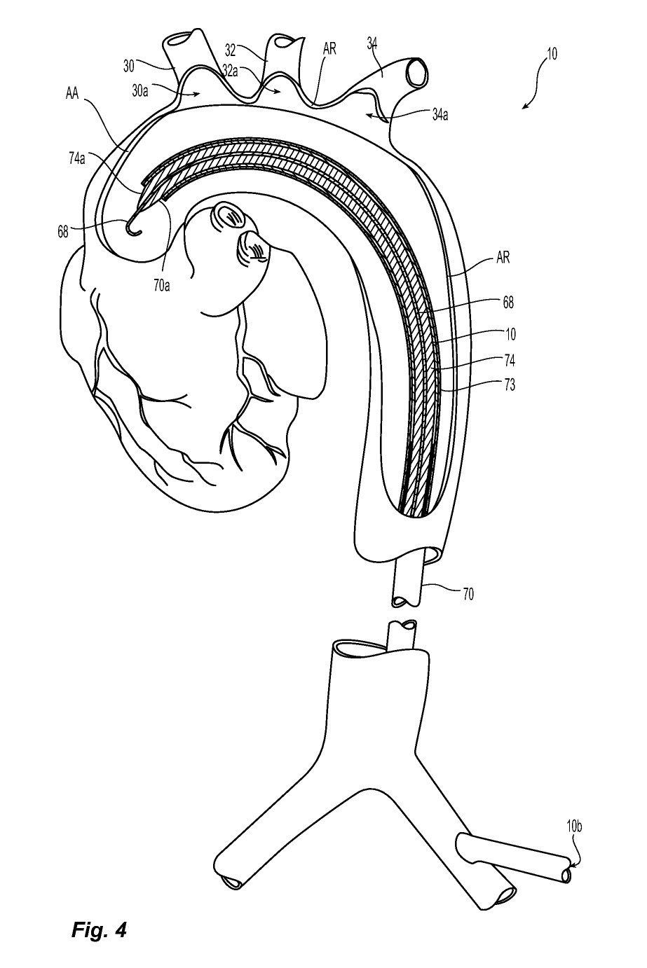

As shown in FIG. 5, an interventional catheter 75, such as a TAVR or a valvuloplasty balloon, can be inserted through the lumen of the EPD 10 to a target location for temporary deployment and/or implantation. The distal terminus of the EPD 10 is accordingly open to enable the TAVR to pass out of the EPD 10. As the interventional cardiac procedure takes place, embolic material is redirected and/or captured within a space 71 between the EPD 10 and the interventional catheter 75, including within and along the length of the embolic protection device 10. During the collection of embolic material, filtered blood continues to perfuse the branching vessels of the aorta.

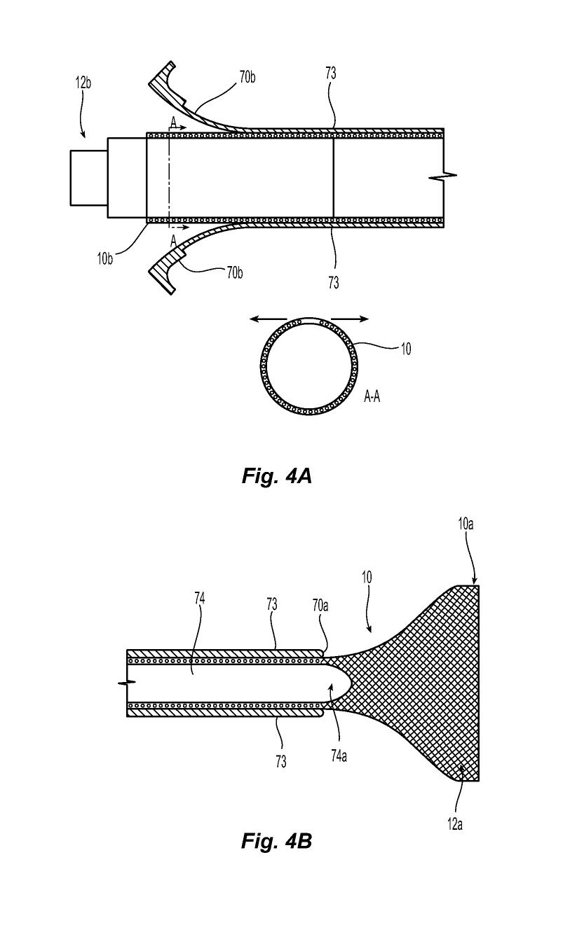

After completing the interventional procedure, the interventional catheter is removed in a proximal (e.g., downstream) direction through the lumen of the EPD 10 and then the EPD is removed from the patient. FIG. 6 is an isometric view of a retrieval sheath 80 for removing the EPD 10 from the vasculature. The retrieval sheath 80 can be a tube or a catheter having a slot 82 along all or only a portion of its length, or the retrieval sheath 80 can be a solid tube. To remove the EPD 10 from the arterial system, the retrieval sheath 80 can be coupled to the proximal portion 18 (FIG. 1) of the EPD 10 and may be advanced distally to collapse the EPD 10 before removal through the aorta. In other embodiments, the proximal region 10b of the EPD 10 can be pulled proximally (e.g., downstream) without the retrieval sheath 80 to remove the EPD 10.

### 3. Mesh Characteristics and Manufacture

The filter portion 12 includes at least one mesh material or layer. In selected embodiments, the mesh may comprise a braided material of filaments (e.g., wires, threads, sutures, fibers, etc.) configured to form a porous fabric or structure. The filter portion may include two or more layers of mesh materials. In some embodiments, braid filaments of varying diameters may be combined in the same layer or portions of the layer to impart different characteristics including, e.g., stiffness, elasticity, structure, radial force, pore size, embolic filtering ability, and/or other features. For example, in the embodiment shown in FIG. 7, the braided mesh has a first mesh filament diameter 90 and a second mesh filament diameter 92 smaller than the first mesh filament diameter 90. In some embodiments, the diameter of the braid filaments can be less than about 0.5 mm. In other embodiments, the filament diameter may range from about 0.01 mm to about 0.40 mm.

The filaments of the braided mesh can be arranged in a generally axially elongated configuration when the EPD 10 is within the delivery catheter or the retrieval catheter. Certain embodiments of the filaments have a filament braid angle “a” from about 5 to 45 degrees with respect to the longitudinal axis of the device such that the filaments are angled toward the longitudinal dimension of the EPD 10. In the expanded or deployed configuration, the braid angle a of the filaments can be from 45 to about 85 degrees with respect to the longitudinal axis of the device. The expanded braided mesh can conform to or otherwise contact the vessels without folds along the longitudinal axis. The cross-sectional dimension of the mesh in the expanded state can be from 5 mm to 50 mm, or from 10 mm to 40 mm in selected embodiments. The diameters of the braided mesh within the delivery catheter and within the retrieval catheter be from 2 mm to 15 mm, or from 5 mm to 10 mm in more specific applications.

The mesh can be constructed using metals, polymers, composites, and/or biologic materials. Polymer materials can include Dacron, polyester, polypropylene, nylon, Teflon, PTFE, ePTFE, TFE, PET, TPE, PLA silicone, polyurethane, polyethylene, ABS, polycarbonate, styrene, polyimide, PEBAX, Hytrel, poly vinyl chloride, HDPE, LDPE, PEEK, rubber, latex, or other suitable polymers. Other materials known in the art of elastic implants can also be used. Metal materials can include, but are not limited to, nickel-titanium alloys (e.g. Nitinol), platinum, cobalt-chrome alloys, 35N LT, Elgiloy, stainless steel, tungsten or titanium. In certain embodiments, metal filaments may be highly polished or surface treated to further improve their hemocompatibility. In some embodiments, it is desirable that the mesh be constructed solely from metallic materials without the inclusion of any polymer materials, i.e., polymer free. In these embodiments and others, it is desirable that the entirety of the embolic protection device be made of metallic materials free of any polymer materials. It is believed that the exclusion of polymer materials in some embodiments may decrease the likelihood of thrombus formation on device surfaces, and it is further believed that the exclusion of polymers and the sole use of metallic components can provide an embolic protection device with a thinner profile that can be delivered with a smaller catheter as compared to devices having polymeric components.

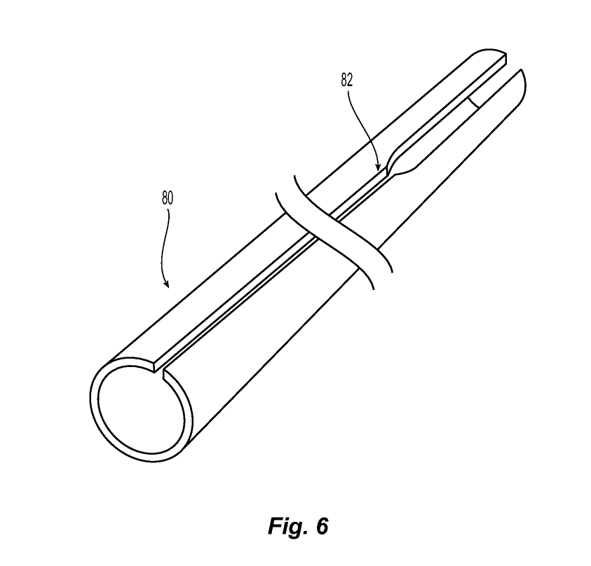

FIG. 8 shows a braided mesh being formed over a mandrel as is known in the art of tubular braid manufacturing. The braid angle alpha a can be controlled by various means known in the art of filament braiding. The braids for the mesh components can have a generally constant braid angle over the length of a component or can be varied to provide different zones of pore size and radial stiffness (as discussed herein). The tubular braided mesh can then be further shaped using a heat setting process. Referring to FIG. 8, as is known in the art of heat setting a braiding filament, such as Nitinol wires, a fixture, mandrel or mold can be used to hold the braided tubular structure in its desired configuration while subjected to an appropriate heat treatment such that the resilient filaments of the braided tubular member assume or are otherwise shape-set to the outer contour of the mandrel or mold. The filamentary elements of a mesh device or component can be held by a fixture configured to hold the device or component in a desired shape and, in the case of Nitinol wires, heated to about 475-525° C. for about 5-30 minutes to shape-set the structure. Such braids of shape memory and/or elastic filaments are herein referred to as “self-expanding.” Other heating processes are possible and will depend on the properties of the material selected for braiding.

The terms “formed,” “preformed” and “fabricated” may include the use of molds or tools that are designed to impart a shape, geometry, bend, curve, slit, serration, scallop, void, hole in the elastic, superelastic, or shape memory material or materials used in the components of the embolic protection device, including the mesh. These molds or tools may impart such features at prescribed temperatures or heat treatments.

For braided portions, components, or elements, the braiding process can be carried out by automated machine fabrication or can also be performed by hand. For some embodiments, the braiding process can be carried out by the braiding apparatus and process described in U.S. Pat. Publication No. 8,261,648, filed Oct. 17, 2011 and entitled “Braiding Mechanism and Methods of Use” by Marchand et al., which is herein incorporated by reference in its entirety. In some embodiments, a braiding mechanism may be utilized that comprises a disc defining a plane and a circumferential edge, a mandrel extending from a center of the disc and generally perpendicular to the plane of the disc, and a plurality of actuators positioned circumferentially around the edge of the disc. A plurality of filaments are loaded on the mandrel such that each filament extends radially toward the circumferential edge of the disc and each filament contacts the disc at a point of engagement on the circumferential edge, which is spaced apart a discrete distance from adjacent points of engagement. The point at which each filament engages the circumferential edge of the disc is separated by a distance “d” from the points at which each immediately adjacent filament engages the circumferential edge of the disc. The disc and a plurality of catch mechanisms are configured to move relative to one another to rotate a first subset of filaments relative to a second subset of filaments to interweave the filaments. The first subset of the plurality of filaments is engaged by the actuators, and the plurality of actuators is operated to move the engaged filaments in a generally radial direction to a position beyond the circumferential edge of the disc. The disc is then rotated a first direction by a circumferential distance, thereby rotating a second subset of filaments a discrete distance and crossing the filaments of the first subset over the filaments of the second subset. The actuators are operated again to move the first subset of filaments to a radial position on the circumferential edge of the disc, wherein each filament in the first subset is released to engage the circumferential edge of the disc at a circumferential distance from its previous point of engagement.

### 4. Filter Portion Layering

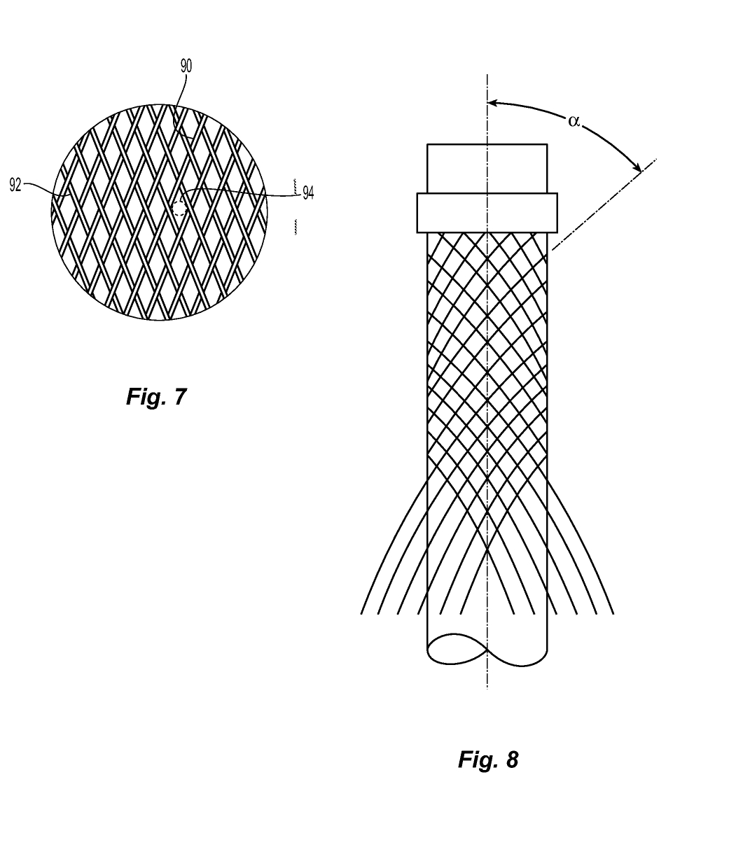

The filter portion can have one or more braids along its whole length or only a portion of its length. In several embodiments, the filter portion has only a single mesh layer, but in other embodiments the filter portion has a plurality of the same or different layers of mesh material. FIG. 9, for example, is a partial cross-sectional view of an embodiment of the filter portion 12 that includes one or more structural braid(s) 100 with a large effective pore size and one or more filtering braid(s) 102 with a substantially smaller effective pore size configured to separate and retain embolic matter relative to the blood flow. The pore size of the structural braid 100 can be greater than 0.20 mm, and generally more than 0.25 mm. The structural braid 100 or portions of the structural braid 100 are configured to provide stability and exert radial forces that secure and shape other layers and/or braids of the filter portion 12 to surrounding tissue structures. The radial force exerted by the structural braid 100 is generally sufficient to inhibit movement, dislodgement and potential embolization of the EPD 10. The structural braid 100 can include one or more of a resilient material, shape memory material, or superelastic material such as Nitinol, for example.

The filtering braid 102 can have small pores that filter and/or retain the emboli. The filtering braid 102, for example, can be a braid with an average effective pore size between about 0.05 mm and about 0.25 mm. The ratio of the effective pore size of the structural braid 100 to the filtering braid 102 can be between about 1.5 and 6. The difference between the effective pore size of the structural braid 100 and the effective pore size of the filtering braid 102 can be between about 0.100 and 0.800 mm. The effective pore size can be determined by measuring more than about 5 pores around the periphery of the EPD 10 where the pores tend to reach a maximum and averaging the numbers.

The filtering braid 102 and the structural braid 100 may have different braid counts. In some embodiments, the braided filament count for the filtering braid 102 is greater than 290 filaments per inch. In one embodiment, the braided filament count for the filtering braid 102 is between about 360 to about 780 filaments per inch, or in further embodiments between about 144 to about 290 filaments per inch. In one embodiment, the braided filament count for the structural braid 100 is between about 72 and about 144 filaments per inch, or in other embodiments between about 72 and about 162 filaments per inch. In some embodiments, the device 100 may include polymer filaments or fabric within the braid(s) 100, 102 or between layers of braids.

The filtering braid 102 and the structural braid 100 may also be comprised of braided filaments having different diameters. For example, in some embodiments, the filtering braid 102 comprises filaments having an average diameter less than 0.04 mm, and the structural braid 100 can have filaments with an average diameter from about 0.07 mm to about 0.20 mm. In other embodiments, the filtering braid 102 comprises filaments having an average diameter of 0.025 mm. In addition, the ratio of the average diameters of the filaments of the structural braid 100 to the average diameters of the filaments of the filtering braid 102 can be from 2:1 to 12:1. In some embodiments, the thickness of the filaments of the structural braid 100 are less that about 0.5 mm. For example, the structural braid 100 may be fabricated from wires or filaments having diameters ranging from about 0.015 mm to about 0.25 mm. In some embodiments, the thickness of the braid filaments of the filtering braid 102 are less that about 0.25 mm. In further embodiments, the structural braid 100 and/or the filtering braid 102 can comprise braids having mixed filament diameters (e.g., thickness).

In the embodiment shown in FIG. 9, the structural braid 100 comprises an innermost layer of the mesh. By placing the structural braid 100 within the filtering braid 102, the structural braid 100 can exert an outward radial force for facilitating a tight fit between the EPD 10 and arterial wall. In some embodiments, the EPD 10 may have a portion or region that is in intimate contact with the arterial wall along substantially all of its length. In other embodiments, the structural braid 100 may alternatively comprise the outermost layer. In yet other embodiments, both mesh layers may be structural braids 100 and/or filtering braids 102. In a different arrangement, the structural and filtering braids 100, 102 can be combined into a single interwoven braid or braid layer that includes all the functions of both the structural and filtering braids 100, 102. Optionally, the filter portion 12 and/or EPD 10 may include other braids or layers in addition to the structural and filtering braids 100, 102. For example, a fabric or polymer layer (e.g., comprising Dacron®, polyester, polypropylene, nylon, Teflon®, or other polymer, fabrics, braids, or knits) can be incorporated into the braid of the filter portion 12 and/or EPD 10.

The braid angle a of the structural braid 100 can be approximately the same as the braid angle a of the filtering braid 102 at corresponding points along the length of the device. In one embodiment, the braid angles of the structural braid 100 and the filtering braid 102 can vary together along the length L of the filter. For example, the filter can have a first region R1 with a first braid angle α₁ and a second region R2 with a second braid angle α₂ that is different than α₁. Within the first region R1, the structural braid 100 and the filtering braid 102 both have approximately the first braid angle α₁. Within the second region R2, the structural braid 100 and the filtering braid can both have approximately the second braid angle α₂, which is different than the first braid angle α₁. Although the embodiment shown in FIG. 9 has two regions, the filter can have more or less than two regions along its length L. Likewise, in other embodiments, the braid angle α may change continuously along its length. In some embodiments, there may be a transition region RT between two braid angle regions. Increasing the braid angle a effectively increases the regional pore size and thus enhances radial stiffness of the device and thus the radial force when contacting the vessel wall. Many embodiments, therefore, can have a higher braid angle a at the distal region 12a of the filter to provide additional anchoring support compared to more proximal regions.

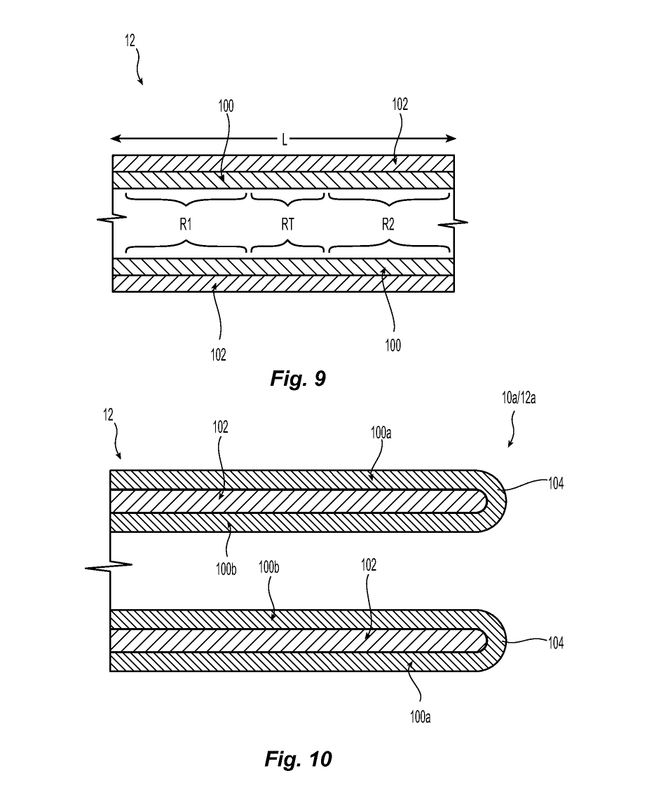

FIG. 10 shows another embodiment in which the structural braid 100 has an outer layer 100a and an inner layer 100b. For example, the structural braid 100 can be folded back on itself (e.g., everted), and the filtering braid 102 may be interposed between the outer layer 100a and the inner layer 100b. The braids 100 and 102 can thus be configured in a substantially coaxial fashion.

In embodiments with multiple layers of braids, the layers or some of the layers can be held at one or more ends by a common connecting member or hub, while the other end is a free end that is not held by a connecting member or hub. The free ends of the braid layers enable the layers to have different lengths without bunching of the layers upon collapse for delivery or retraction by a catheter because the layers can move relative to each other to accommodate compression into a contracted state. The characteristics of the structural layer or braid material can remain constant as the braid continues around the everted portion at an edge 104, or it can be formed with two or more braiding techniques so that the braiding on the inside for the inner layer 100b is different than the braiding on the outside for the external layer 100a. For example, the braiding can change to provide differing braid angles or pore sizes.

From the foregoing, it will be appreciated that specific embodiments of the invention have been described herein for purposes of illustration, but that various modifications may be made without deviating from the scope of the invention. Accordingly, the invention is not limited except as by the appended claims.

1. An embolic protection device for placement in an arterial system of a human, the arterial system having an ascending aorta, an aortic arch, and a descending aorta, wherein a brachiocephalic artery ostium and at least one of a left common carotid artery ostium and a left subclavian artery ostium branch from the aortic arch, comprising:
a filter having a delivery configuration and a deployed configuration, the filter having—
a first filter section having a first cross-sectional dimension in the deployed configuration sufficient to anchor the embolic protection device within the ascending aorta and to provide passage of a therapeutic device through the first filter section; and
a tapered second filter section extending proximally from the first filter section; and
a proximal portion extending proximally from the second filter section to an extracorporeal location.

2. The embolic protection device of claim 1 wherein the first filter section in the deployed configuration is configured to contact the aortic arch at the brachiocephalic artery ostium and at least one of the left common carotid artery ostium or the left subclavian artery ostium.

3. The embolic protection device of claim 1 wherein the filter further comprises:
a structural braid having a first pore size; and
a filtering braid having a second pore size smaller than the first pore size.

4. The embolic protection device of claim 1 wherein the first filter section is configured to contact a full circumference of an inner wall of the aortic arch from the ascending aorta to an area downstream of the left subclavian artery.

5. The embolic protection device of claim 1 wherein the filter further comprises a braid angle that varies along the length of the filter.

6. The embolic protection device of claim 1 wherein the first filter section is configured to contact a full circumference of an inner wall of the aortic arch from the ascending aorta to the descending aorta.

7. The embolic protection device of claim 1 wherein the proximal portion further comprises a first proximal section and a second proximal section, wherein the first proximal section comprises a coated mesh and the second proximal section comprises an expandable tube.

8. The embolic protection device of claim 1 wherein the proximal portion comprises a mesh.

9. The embolic protection device of claim 1 wherein the first filter section has a first pore size and the second filter section has a second pore size different than the first pore size.

10. The embolic protection device of claim 1 wherein the first filter section has a first braid angle and the second filter section has a second braid angle

11. (canceled)

12. The embolic protection device of claim 1 wherein the filter further comprises:
a first mesh layer having a first pore size;
a second mesh layer having a second pore size;
a third mesh layer between the first mesh layer and the second mesh layer, the third mesh layer having a third pore size; and
wherein the first pore size and the second pore size are individually less than the third pore size.

13. The embolic protection device of claim 1 wherein the filter further comprises:
a first area having a first braid angle;
a second area having a second braid angle less than the first braid angle;
wherein the first area is located at a distal zone of the first filter section and is configured to contact a portion of the ascending aorta distal to the brachiocephalic artery ostium when the embolic protection device is deployed.

14. The embolic protection device of claim 1 wherein the second filter section further comprises a second cross-sectional dimension less than the first cross-sectional dimension.

15. The embolic protection device of claim 1 wherein the proximal portion comprises a coated mesh.

16. The embolic protection device of claim 1 wherein the proximal portion comprises an expandable tube.

17. The embolic protection device of claim 1 wherein the proximal portion comprises a coated mesh region and an expandable tube region, wherein the coated mesh region is distal the expandable tube region.

18-23. (canceled)

24. A method for providing embolic protection throughout the vasculature of a patient, including the arterial system having an ascending aorta, an aortic arch, and a descending aorta, and ostium from the aortic arch, comprising:
delivering a distal region of an embolic protection device to a portion of the ascending aorta and a proximal region of the device to the descending aorta;
expanding the embolic protection device to contact at least a portion of the arterial system distal to the brachiocephalic artery ostium; and
retaining emboli along a length of the embolic protection device extending from a point distal to the brachiocephalic artery ostium to an extracorporeal location.

25. The method of claim 24, further comprising removing the emboli from the vasculature while the embolic protection device remains expanded.

26. The method of claim 24 wherein expanding the embolic protection device further comprises contacting the brachiocephalic artery ostium, the left common carotid artery ostium, and the left subclavian artery ostium with the device.

27. The method of claim 24 wherein expanding the embolic protection device further comprises contacting the aortic arch from the brachiocephalic artery ostium to the left subclavian artery ostium with the embolic protection device such that a filter portion of the embolic protection device spans across the brachiocephalic artery ostium, the left common carotid artery ostium, and the left subclavian artery ostium.

28. The method of claim 24, further comprising redirecting emboli through a lumen of the embolic protection device to an extracorporeal location.

29. The method of claim 24 wherein expanding the embolic protection device further comprises spanning the brachiocephalic artery ostium, the left common carotid artery ostium, and the left subclavian artery ostium with a filter portion of the embolic protection device.

30. The method of claim 24 wherein expanding the embolic protection device further comprises contacting a portion of the aortic arch with a filter portion of the embolic protection device between the brachiocephalic artery ostium and the left common carotid artery ostium and between the left common carotid artery ostium and the left subclavian artery ostium.

31. The method of claim 24 wherein capturing the emboli further comprises trapping the emboli between a first mesh layer and a second mesh layer of the device.

32. The method of claim 24 wherein capturing the emboli further comprises trapping the emboli between a first mesh layer of the embolic protection device and a second mesh layer of the embolic protection device, and wherein the method further includes simultaneous removal of trapped emboli and the embolic protection device from the vasculature.

33-37. (canceled)
